(19) United States
(12) Patent Application Publication
(10) Pub. No.: US 2022/0088315 A1
(43) Pub. Date: Mar. 24, 2022

(54) DELIVERY SYSTEM AND METHOD FOR SUCH DELIVERY SYSTEM

(71) Applicant: GO-PEN APS, Ålsgårde (DK)

(72) Inventors: Ole Kjerkegaard Nielsen, Ålsgårde (DK); Michael Ejstrup Hansen, Morud (DK)

(21) Appl. No.: 17/424,439

(22) PCT Filed: Jan. 16, 2020
(86) PCT No.: PCT/EP2020/051074

§ 371 Date: Jul. 20, 2021

(30) Foreign Application Priority Data
Jan. 23, 2019 (DK) PA201900097

(51) Int. Cl. A61M 5/315; A61M 5/28

(52) U.S. Cl. CPC A61M 5/31536; A61M 5/31515; A61M 5/3158; A61M 2005/3114; A61M 5/281; A61M 2205/583; A61M 5/31553

(57) ABSTRACT

A delivery system includes a cartridge storing a liquid, and a dosing mechanism being configured to connect to the cartridge and to dispense a predetermined amount of liquid from the cartridge. The cartridge is non-reversibly transformable from a filling configuration, in which liquid can be drawn into the cartridge, to a dispensing configuration, in which liquid can only be dispensed from the cartridge

### CROSS-REFERENCE TO RELATED APPLICATIONS

This application is the U.S. National Stage of PCT/EP2020/051074 filed Jan. 16, 2020, which claims priority to Denmark Application No. PA 201900097, filed Jan. 23, 2019, the entire content of both are incorporated herein by reference in its entirety.

### FIELD OF THE INVENTION

The present invention relates to the technical field of medication dosage. More specifically, the present invention relates to providing a safe, convenient and cheap system for medication dosage. In particular, the system and method of the present application relate to an alternative to the current syringe/needle solution that is common for vial users. A delivery system and method for such delivery system, are disclosed and claimed herein.

### BACKGROUND OF THE INVENTION

Most syringes in use today are of the disposable or single-use type. A typical disposable syringe is made primarily of plastic and has several key components. The largest, and the one containing the most material, is the plastic barrel. The scale printing on the barrel is a critical and costly assembly step that is needed to assure proper dosing by the user. Inside the barrel is typically a rubber stopper that is used to create a hermetic seal and to displace the liquid medication or other fluid into and out from the barrel. The rubber stopper determines the correct dosage upon extracting the liquid from a container. A plastic rod interfaces with the rubber stopper to move it back and forth by means of the user's control. A metal needle or cannula is usually attached to the distal end of the barrel to allow fluids to be injected into the body, or removed from the body. After a single use the entire syringe is disposed of, and none of the components is reused.

When it comes to diabetes, disposable plastic syringes are often used to administer liquid insulin to a user several times a day. These single-use syringes typically have transparent plastic barrels with printed scale numbers that allow the user to draw up an accurate dose of insulin from a vial, and fine-gauge metal needles that inject the dose into the body of the patient. Insulin syringes usually have a capacity of 1 ml or less (with 0.3 ml, 0.5 ml and 1.0 ml barrel sizes being common), with scale markings on the barrel representing units of a specific type of insulin (e.g., U-100 or U-S00 insulin). Insulin syringes may also be provided with safety features to prevent reuse of the syringe, to shield the used needle, or both. Because insulin syringes are used only once and a user usually requires several of them each day, they are commonly sold in boxes or bags containing multiple syringes.

The cheapest form of treatment for a variety of chronic diseases where patients should have daily injections is today performed by using a standard disposable syringe as described above, in combination with a vial. Typically, patients who use the cheapest form of treatment will themselves purchase or receive a vial of medication. They will accordingly by themselves arrange the medicine into the syringe and subsequently inject the contents of the syringe.

The treatment procedure of today has a number of disadvantages, relating to personal safety and cost, in particular. Firstly, producing the correct amount of dosage has been a known drawback since the introduction of personally injectable medication systems. Individuals suffering from chronic diseases do not necessarily possess the required knowledge behind a correct medication dosage. This may cause severe side effects resulting in hazardous situations, and in worst-case, fatalities. Moreover, today's injectable medication systems are not very convenient. A user will normally require carrying around several syringes a day, every day, to ensure safety. This of course is a bothersome activity. Another factor of concern is the cost of the disposable syringes. As they are not reusable, the accumulated cost will quickly ramp up causing certain individuals to have their lives economically affected. Finally, one could consider the disposable syringes as being environmentally unfriendly.

The present inventors have registered and analyzed the problems and shortcomings regarding the aforementioned aspects. Accordingly, an object of the present invention is therefore to overcome or at least mitigate one of these problems.

### SUMMARY OF THE INVENTION

It is an object of the invention to at least partly overcome one or more of the above-identified limitations of the prior art. By introducing a syringe that can be used as a cartridge to be inserted into a dosing system after being filled from a larger container, such as a vial, today's users of standard syringes and vials will be given an opportunity to combine the cheap vial-based administration with safe and robust handling.

An object of the present invention is therefore to provide one or more improvements when it comes to injectable medication devices, i.e. to delivery systems.

According to a first aspect of the invention, a delivery system is provided. The delivery system comprises a cartridge storing a liquid, and a dosing mechanism being configured to connect to the cartridge and to dispense a predetermined amount of liquid from said cartridge. The cartridge is non-reversibly transformable from a filling configuration, in which liquid can be drawn into the cartridge, to a dispensing configuration, in which liquid can only be dispensed from the cartridge.

In an embodiment, the cartridge can be connected to the dosing mechanism only when the cartridge is in the dispensing configuration. This ensures proper use of the cartridge.

The dosing mechanism may have a first end to which the cartridge can be connected. Facilitated mounting of the cartridge to the dosing mechanism is thereby provided.

The dosing mechanism may further comprise an opposite end being provided with an injection control means. Preferably, the injection control means is a manual control means. The dosing mechanism thereby allows a user to easily maneuver the dosing mechanism, such that the correct amount of medication is discharged.

The manual injection control means may comprise a control button.

In an embodiment the dosing mechanism is configured to control dispensing of a specific volume of liquid from the cartridge. The specific volume is preferably less than the total volume of liquid stored inside the cartridge. Hence a single cartridge can be used for multiple medication dosing.

The specific volume may be adjustable. For this purpose, the dosing mechanism may comprise a quantity control device being configured to adjust the specific volume being dispensed from the cartridge. In an embodiment, the quantity control device is provided as a turn knob. By turning the knob, the user can thereby change the volume to be discharged upon operation of the dosing mechanism.

The dosing mechanism may further comprise a visual indicator, in order to show the current setting for the specific volume to a user.

The cartridge may be provided with a needle adaptor at one end thereof.

In an embodiment, the cartridge is in the form of a disposable syringe. The cartridge may comprise a container and a piston arranged inside the container, wherein motion of the piston is used to draw liquid into the container as well as dispense liquid out from the container.

The cartridge may be connectable to a piston rod which can be manually maneuvered in order to fill the cartridge from e.g. a vial.

The piston rod may be releasably connected to the piston, such that the piston rod is disconnected from the piston when the piston is arranged in a position where the container of the cartridge has been filled.

The cartridge may be distributed to a user in its filling configuration. In the filling configuration, the piston rod is preferably connected to the piston.

The piston rod may be automatically released from the piston when the cartridge is filled with liquid. Alternatively, the piston rod is configured to be manually released from the piston when the cartridge is filled with liquid.

In an embodiment, the piston rod is connected to the piston when the cartridge is in the filling configuration. The piston rod is disconnected from the piston when the cartridge is in the dispensing configuration.

The piston rod may be connected to the piston by means of a plurality of arms. The arms may be resilient. In an embodiment, the arms are biased outwards but flexible inwards such that the arms may fit inside the container of the cartridge.

The arms may be configured to grip a button when the arms are arranged inside the container. The arms may be configured to release the button when the arms are arranged outside the container.

According to a second aspect, a cartridge is provided. The cartridge is configured to form part of a delivery system according to the first aspect.

According to a third aspect, a dosing mechanism is provided. The dosing mechanism is configured to form part of a delivery system according to the first aspect.

According to a fourth aspect, a method for providing a delivery system is provided. The method comprises a first step of providing a cartridge, a second step of filling the cartridge with a certain amount of liquid, a third step of transforming the cartridge from a filling configuration to a dispensing configuration, and a step of loading the cartridge to a dosing mechanism.

### BRIEF DESCRIPTION OF THE DRAWINGS

Embodiments of the invention will now be described, by way of example, with reference to the accompanying schematic drawings, in which

FIG. 1 is an isometric view of a delivery system according to an embodiment, in a ready-to-use state;

FIG. 2 is an isometric view of different parts forming the delivery system shown in FIG. 1;

FIG. 3 is a schematic view of a method for providing a delivery system according to an embodiment;

FIG. 4 is an isometric view of a disposable cartridge according to an embodiment;

FIG. 5 is an isometric view of the disposable cartridge shown in FIG. 4, in a state prior to forming the delivery system; and

FIGS. 6a-b are isometric views of details of a disposable cartridge according to an embodiment.

### DETAILED DESCRIPTION OF PREFERRED EMBODIMENT

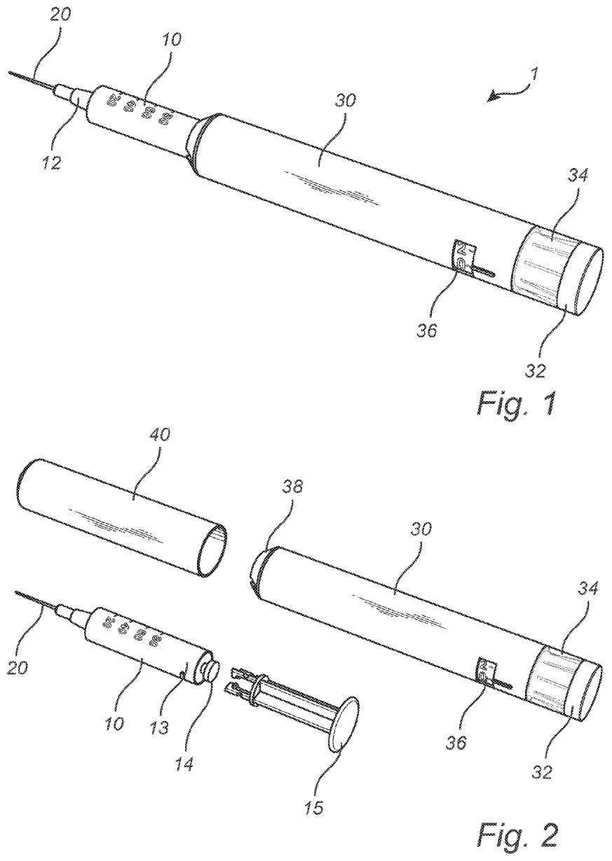

Starting in FIG. 1, a delivery system 1 is shown. The delivery system 1 is configured to administrate injections by delivering a pre-determined amount of a specific medication to a user. The medication is stored in a cartridge 10, having a needle adapter 12 at its open end. As can be seen in FIG. 1, the cartridge 10 is further connected to a needle 20 by means of the needle adaptor 12. The delivery of medication through the needle 20, from the cartridge 10 to a user, is controlled by means of a dosing mechanism 30. The dosing mechanism 30 is loaded with the cartridge 10, and the dosing mechanism 30 further comprises an injection control means 32. The injection control means 32 can be manually operated, and in the shown example, the manual injection control means 32 is in the form of a button, whereby depressing the button 32 will cause dispensing of medication from the cartridge 10 through the needle 20.

The exact amount of medication being injected upon activation of the delivery system 1, i.e. by a user pressing the button 32, is preferably adjustable. For this, the dosing mechanism 30 may be provided with a quantity control device 34, here in the form of a turn knob. By rotating the turn knob, the stroke of the dosing mechanism 30 is adjusted thereby also controlling the exact amount of medication delivery.

As can be further seen in FIG. 1, the dosing mechanism 30 is provided with a visual indicator 36 in the form of quantity meter. As the quantity control device 34 is operated, by rotating the turn knob, the visual indicator 36 will show the current setting, i.e. how much medication will be administrated. The current setting of the quantity control device 34 is preferably always visible by means of the visual indicator 36.

The exact configuration of the dosing mechanism 30 may vary, as long as it is configured to receive a cartridge 10 and to allow for controlled injection of a predetermined part of the enclosed volume of medication from the cartridge 10.

The different parts used to form the delivery system 1 are further shown in FIG. 2. The dosing mechanism 30 is provided as an open-ended tube, being provided with the control button 32 at one closed end thereof. The opposite end 38 is ready to receive a filled cartridge 10. Loading the cartridge 10 into the dosing mechanism 30 may be done in various ways, for example by a snap-fit connection, by a threaded interface, by a separate locking function, etc. Importantly, the dosing mechanism 30 should allow for easy and safe loading and unloading of the cartridge 10. The cartridge 10 may not necessarily be loaded at the end 38 of the dosing mechanism 30, but the dosing mechanism 30 may be provided with a slot or other receiving structure capable of receiving and connecting the cartridge 10.

The dosing mechanism 30 is further provided with a sleeve-like protective cover 40. The protective cover 40 can be slid onto the connected cartridge 10, and connect to the end 38 of the dosing mechanism 30 preferably by means of friction.

The cartridge 10 is also shown in further details in FIG. 2. In particular, the cartridge 10 comprises a container 13 in the form of a barrel, in which a piston 14 is mounted. The piston 14 can be coupled to a piston rod 15, although in FIG. 2 the piston rod 15 is shown in a disconnected state. According to the embodiments described herein, the piston rod 15 can be released from the piston 14 when the container 13 is sufficiently filled with liquid, which allows the cartridge 10 to be coupled with the dosing mechanism 30 after filling from a vial or similar.

In fact, the dosing mechanism 30 is preferably designed such that it cannot connect to the cartridge 10 when the cartridge 10 is in a state where it is configured to draw liquid from a vial or similar. Instead, the dosing mechanism 30 requires the cartridge 10 to transform to another state after it has been filled, in order for it to be properly loaded into the dosing mechanism 30.

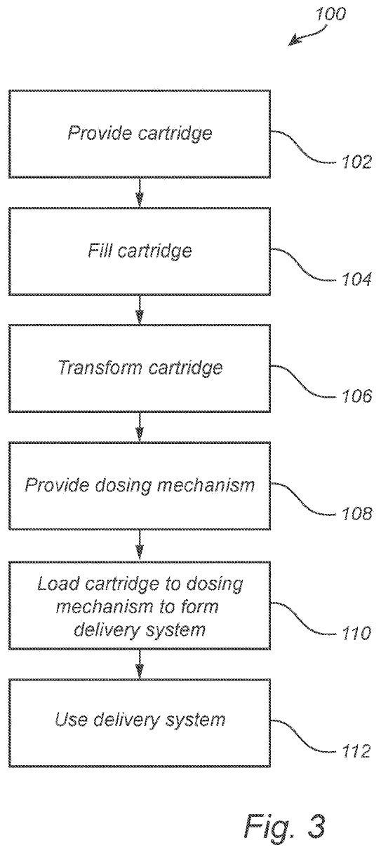

In FIG. 3, a method 100 for providing a delivery system 1 is schematically shown. As will be clear from the following description, the method 100 is based on the engagement of the different parts described earlier with reference to FIGS. 1 and 2.

In a first step 102, a cartridge 10 is provided. The cartridge 10 is preferably provided in a ready-to-fill state, such that a user may easily maneuver the cartridge in the next step 104, which is performed to fill the cartridge 10 with a certain amount of liquid. After filling of the cartridge 10, the cartridge 10 is transformed from a filling state, or configuration, to a loading state, or dispensing configuration, in a transformation step 106. This transformation is preferably performed automatically when the cartridge has been completely filled, or as a final sequence of the filling step 104.

At this point (or earlier, before or in parallel with any of the previously described steps 102-106), the dosing mechanism 30 is provided in a step 108. Once the dosing mechanism 30 is provided, the cartridge 10 is loaded to the dosing mechanism 30 in a step 110. At this point, the delivery system 1 is formed. In a final step 112 the delivery system 1 is used to inject medication to a user. This step may be preceded by a step of connecting a needle 20 to the cartridge of the delivery system 1. The final step 112 of using the delivery system 1 can be repeated multiple times; the cartridge 10 is preferably filled by an amount being a multiple of the normal dose volume, so that the same cartridge 10 can be used for several injections before it is emptied and needs to be replaced.

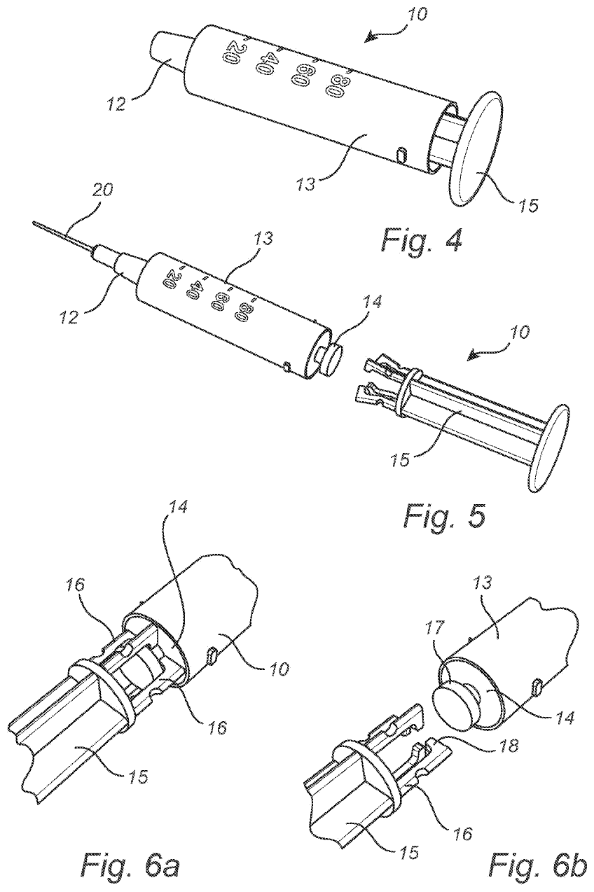

In FIGS. 4-6 further details of the cartridge 10 are shown. In FIG. 4 the cartridge 10 is shown in a ready-to-fill state (although a needle 20 is typically required to draw liquid from an associated vial). This is preferably the state in which the cartridge 10 is distributed to a user. As is shown, the piston (which at this point is arranged inside the cartridge 10), as well as the piston rod 15, are inserted into the barrel 13. Typically the user operates the cartridge 10 by connecting a needle (not shown) to the cartridge 10 via the needle adaptor 12 (see FIG. 5), and draws medication into the barrel 13 by inserting the needle 20 into a vial and by pulling the piston rod 15 away from the barrel 13. Once the barrel 13 is filled with medication, the piston 14 will reach an uppermost position at which the piston rod 15 will be released from the piston 14, and hence also from the cartridge 10. This is shown in FIG. 5. Preferably, the piston rod 15 is automatically released from the piston 14 when the piston 14 reaches its uppermost position.

The cartridge 10 is preferably made of a transparent, or at least partly transparent material. A quantity indicator in the form of pre-printed labels and levels is used to allow a user to monitor the amount of content inside the barrel 13.

As been explained previously, the piston rod 15 is preferably automatically released from the cartridge 10 when the cartridge 10 has been completely filled with liquid. One embodiment of a suitable connection of the piston rod 15, in order to achieve automatic detachment, is shown in FIGS. 6a-b. One end of the piston rod 15 is provided with a plurality of arms 16. The arms 16, being four in the shown example, connect to an end button 17 of the piston 14. The button 17 is disc-like, such that the arms 16 can grip behind the button 17 by means of hooks 18; each arm 16 is for this purpose provided with a hook 18 protruding radially inwards.

Each arm 16 is flexible in the radially outward direction, e.g. by means of a pivot joint somewhere along the length of the respective arms 16. This means that the arms 16 will flex outwards, such that the arms 16 will no longer be able to grip around the button 17 of the piston 14. However, during filling the arms will be prevented to move radially outwards due to the walls of the barrel 13. During filling, the arms 16 will securely grip the piston but as soon as the button 17 of the piston 14 comes out from the barrel 13, the arms 16 will flex outwards such that they release the grip around the button 17. The piston rod 15 is then disconnected from the cartridge 10, and the cartridge 10 cannot be refilled. Once filled, it can preferably only be used by proper loading of the cartridge 10 into an associated dosing mechanism 30.

Some different aspects of different embodiments of a delivery system 1 will now be described. The cartridge 10 may be in the form of a disposable syringe consisting of a container 13 in which a piston 14 is coupled to a piston rod 15. The piston rod 15 can be released from the piston 14 when the container 13 is sufficiently filled with liquid, which ensures that the syringe, or cartridge 10, can be coupled with a dosing mechanism 30 after filling, for example, from a vial.

The container 13 and piston 14 can preferably be automatically separated from the piston rod 15 after filling, so that the filled container 13 can be mounted to the dosing mechanism 30.

In another embodiment, the container 13 and the piston 14 are manually separated from the piston rod 15 by rotation, tilt or the like, so that the filled container 13 can be mounted on the dosing mechanism 30.

The piston 14 is moved during operation of the delivery system 1. Prior to dosing the piston 14 is located at the upper part of the container 14, while after use the piston 14 is placed in the bottom of the container 14.

Preferably, after use the piston 14 is positioned in the bottom of the container 13 so that the piston rod 15 cannot be reassembled and thereby used to operate the piston 14. Hence, the disposable cartridge 10 cannot be reused.

The mounted piston 14 is configured to have a retaining geometry, e.g. by means of the arms 16 and hooks 18, which ensures that the piston rod 15 engagement is robust during filling of the container 13.

The rear edge of the container 13, together with the piston 14 and/or piston rod 15 is configured to create a transition where the piston 14 and piston rod 15 change from being engaged to being separated. During filling, this transition creates the point where the piston rod 15 is preferably automatically separated from the container 13 and from the piston 14. To ensure that the piston's 14 engagement with the piston rod 15 is released, a flexible zone (e.g. by means of the flexible arms 16) can be introduced on the piston 14 and/or piston rod 15 so that the engagement is interrupted.

The piston rod 15 can be disengaged from the piston 14 as a consequence of the coupling being determined by a restriction of the internal diameter of the container 13. The container 13 may further be provided with one or more geometric details which ensure that the cartridge 10 can be coupled to a dosing mechanism. Such geometric detail may e.g. be hooks, flanges, or other structures on the exterior surface of the container 13 forming a connection interface.

In accordance with the specification herein, a disposable syringe is described as a cartridge in a durable injection device. In particular, a syringe is disclosed which can be used in a system for liquid medication dosing, e.g. insulin.

By providing a syringe that can be used as a cartridge in a dosing system after being filled from a larger container, previous users of vials and standard syringes are given the option to connect the cartridge to a dosing system and thereby benefit from better dosing precision as well as the possibility to carry a more convenient and smaller dosing system to their workplace, or similar.

Hence, a disposable syringe is provided. The disposable syringe comprises a container, in which a piston is connected to a piston rod. The disposable syringe is characterized in that the piston rod can be disconnected from the piston when the container is sufficiently filled with a liquid, which thereby ensures that the disposable syringe, after being filled from e.g. a vial, can be connected to a dosing mechanism.

In an embodiment of the disposable syringe, or of the delivery system described above, the container and the piston are automatically disconnected from the piston rod after filling, whereby the filled container can be mounted to a dosing mechanism.

In an embodiment of the disposable syringe, or of the delivery system described above, the container and the piston rod can be manually separated from the piston by rotation, tilting, or similar, whereby the filled container can be mounted to a dosing mechanism.

In an embodiment of the disposable syringe, or of the delivery system described above, the piston, prior to dosing, is arranged at the top of the container and after use, is arranged at the bottom of the container.

In an embodiment of the disposable syringe, or of the delivery system described above, the piston after use is arranged in the bottom position, whereby the piston rod can not be mounted again to the piston and pull the piston, such that the syringe can not be reused.

In an embodiment of the disposable syringe, or of the delivery system described above, the assembled piston has a geometry that ensures a connection with the piston rod during filling of the container.

In an embodiment of the disposable syringe, or of the delivery system described above, the piston rod has a geometry that ensures a connection with the piston during filling of the container.

In an embodiment of the disposable syringe, or of the delivery system described above, the rear edge of the container forms, together with the piston and/or the piston rod, the transition where the piston and piston rod changes from a connected state to a disconnected state; this transition forms a situation during filling where filling is terminated and the piston rod is separated from the container/piston; in order to ensure that the connection of the piston to the piston rod is terminated, a flexible zone is provided on the piston and/or the piston rod, such that connection is terminated at the interface.

In an embodiment of the disposable syringe, or of the delivery system described above, the piston rod can be separated from the piston as a result of the connection being formed by a limitation of the internal diameter of the container.

In an embodiment of the disposable syringe, or of the delivery system described above, the container is provided with one or more geometric features which ensure that the disposable syringe can be connected to a dosing mechanism.

Also, a dosing mechanism is provided. The dosing mechanism is configured to be connected to the disposable syringe described above.

From the description above follows that, although various embodiments of the invention have been described and shown, the invention is not restricted thereto, but may also be embodied in other ways within the scope of the subject-matter defined in the following claims.

1. A delivery system comprising:
a cartridge storing a liquid; and
a dosing mechanism configured to connect to the cartridge and to dispense a predetermined amount of the liquid from said cartridge;
wherein the cartridge is non-reversibly transformable from a filling configuration, in which the liquid can be drawn into the cartridge, to a dispensing configuration, in which the liquid can only be dispensed from the cartridge.

2. The delivery system according to claim 1, wherein the cartridge is connectable to the dosing mechanism only when the cartridge is in the dispensing configuration.

3. The delivery system according to claim 1, wherein the dosing mechanism has a first end to which the cartridge is connected.

4. The delivery system according to claim 3, wherein the dosing mechanism further comprises an opposite end being provided with a manual injection control means.

5. The delivery system according to claim 4, wherein the manual injection control means comprises a control button.

6. The delivery system according to claim 1, wherein the dosing mechanism is configured to control dispensing of a specific volume of the liquid from the cartridge, the specific volume being less than a total volume of the liquid stored inside the cartridge, wherein the specific volume is adjustable.

7-8. (canceled)

9. The delivery system according to claim 6, wherein the dosing mechanism comprises a quantity control device being configured to adjust the specific volume being dispensed from the cartridge, wherein the quantity control device is provided as a turn knob.

10. (canceled)

11. The delivery system according to claim 1, wherein the dosing mechanism further comprises a visual indicator.

12. The delivery system according to claim 1, wherein the cartridge is provided with a needle adaptor at one end thereof.

13. The delivery system according to claim 1, wherein the cartridge is in the form of a disposable syringe. A delivery system includes a cartridge storing a liquid, and a dosing mechanism being configured to connect to the cartridge and to dispense a predetermined amount of liquid from the cartridge. The cartridge is non-reversibly transformable from a filling configuration, in which liquid can be drawn into the cartridge, to a dispensing configuration, in which liquid can only be dispensed from the cartridge

14. The delivery system according to claim 1, wherein the cartridge comprises a container and a piston arranged inside the container, wherein motion of the piston is used to draw the liquid into the container as well as dispense the liquid out from the container, wherein the cartridge is connectable to a piston rod which is manually maneuverable, the piston rod being releasably connected to the piston.

15-17. (canceled)

18. The delivery system according to claim 14, wherein:
in the filling configuration, the piston rod is connected to the piston; and
the piston rod is;
automatically released from the piston when the cartridge is filled with the liquid; or
configured to be manually released from the piston when the cartridge is filled with the liquid.

19-20. (canceled)

21. The delivery system according to claim 14, wherein:
the piston rod is connected to the piston when the cartridge is in the filling configuration; and/or
the piston rod is disconnected to the piston when the cartridge is in the dispensing configuration.

22. (canceled)

23. The delivery system according to claim 14, wherein the piston rod is connected to the piston by means of a plurality of resilient arms.

24. (canceled)

25. The delivery system according to claim claim 23, wherein:
the arms are biased outwards but flexible inwards such that the arms fit inside the container of the cartridge.

26. The delivery system according to claim 25, wherein:
the arms are configured to grip a button when the arms are arranged inside the container;
and/or
the arms are configured to release the button when the arms are arranged outside the container.

27. (canceled)

28. A cartridge, being configured to form part of a delivery system according to claim 1.

29. A dosing mechanism, being configured to form part of a delivery system according to claim 1.

30. A method for providing a delivery system, comprising:
a first step of providing a cartridge;
a second step of filling the cartridge with a certain amount of liquid;
a third step of transforming the cartridge from a filling configuration to a dispensing configuration; and
a step of loading the cartridge to a dosing mechanism.

31. The delivery system according to claim 1, wherein the cartridge is non-reversibly transformed at an end of a filling with the liquid.
